(19) United States
(12) Patent Application Publication
(10) Pub. No.: US 2012/0116307 A1
(43) Pub. Date: May 10, 2012

(54) DIFFUSION DELIVERY SYSTEMS AND METHODS OF FABRICATION

(75) Inventors: Mauro Ferrari, Houston, TX (US); Xuewu Liu, Westerville, OH (US); Piyush Mohan Sinha, Beaverton, OR (US); Bryan Smith, Columbus, OH (US); Sadhana Sharma, Waukegan, IL (US)

(73) Assignee: THE OHIO STATE UNIVERSITY, Columbus, OH (US)

(21) Appl. No.: 13/289,177

(22) Filed: Nov. 4, 2011

Related U.S. Application Data
(63) Continuation of application No. 11/278,812, filed on Apr. 5, 2006.
(60) Provisional application No. 60/668,468, filed on Apr. 5, 2005.

(51) Int. Cl. A61M 5/00

(52) U.S. Cl. 604/131

(57) ABSTRACT

The invention generally relates to diffusion delivery systems and more particularly to high precision nanoengineered devices for therapeutic applications. The device contains diffusion areas that may be fabricated between bonded substrates, and the device can possess high mechanical strength. The invention further relates to capsules containing a diffusion delivery system. The present invention also relates to methods of fabricating the diffusion delivery systems.

### CROSS REFERENCE TO RELATED APPLICATIONS

This application claims priority to and any other benefit of U.S. Provisional Application Ser. No. 60/668,468, filed Apr. 5, 2005, the entire content of which is incorporated by reference herein.

### BACKGROUND OF THE INVENTION

Considerable advances have been made in the field of drug delivery technology over the last three decades, resulting in many breakthroughs in clinical medicine. However, important classes of drugs have yet to benefit from these technological successes. The creation of drug delivery devices that are capable of delivering therapeutic agents that cannot be delivered by any other means or that have diminishment of therapeutic efficacy when given by other means of administration is a challenge in this area of research. One of the major requirements for an implantable drug delivery device is controlled release of therapeutic agents, especially biological molecules, as a continuous delivery over an extended period of time. The goal here is to achieve a continuous drug release profile consistent with zero-order kinetics where the concentration of drug in blood remains constant throughout the delivery period. Another significant challenge in drug delivery is to engineer a delivery system that can deliver a drug in a manipulated non-zero order fashion such as pulsatile or ramp or some other pattern.

These devices have the potential to improve therapeutic efficacy, diminish potentially life-threatening side effects, improve patient compliance, minimize the intervention of healthcare personnel and reduce the duration of hospital stays.

### SUMMARY OF THE INVENTION

In some embodiments, the present invention provides a device comprising a first substrate having a first face and a second substrate having a first face, wherein the first face of the first substrate is proximate to the first face of the second substrate. The first substrate comprises a first flow path having a plurality of first protrusions on the first face of the first substrate, a second flow path having a plurality of second protrusions on the first face of the first substrate, and a plurality of diffusion areas. At least one of the plurality of first protrusions is disposed between a corresponding pair of second protrusions. A diffusion area is disposed between at least one of the plurality of first protrusions and each of the corresponding pair of second protrusions. Each of the plurality of first protrusions have an aspect ratio that allows each of the plurality of first protrusions to fill with a fluid. In some embodiments, each of the plurality of second protrusions have an aspect ratio that allows each of the plurality of second protrusions to fill with a fluid. In some embodiments, the second substrate further comprises at least one electrode. In some embodiments, the second substrate further comprises at least two electrodes. In some embodiments, one of the electrodes is disposed in communication with the first flow path and one of the electrodes is disposed in communication with the second flow path.

In other embodiments, the present invention provides a device comprising a first substrate having a first face and a second substrate having a first face, wherein the first face of the first substrate is proximate to the first face of the second substrate. The first substrate comprises a first flow path having a plurality of first protrusions on the first face of the first substrate, wherein each of the plurality of first protrusions has a depth and a width, a second flow path having a plurality of second protrusions on the first face of the first substrate, wherein each of the plurality of second protrusions has a depth and a width, and a plurality of diffusion areas each of the plurality of diffusion areas having a length and a depth. At least one of the plurality of first protrusions is disposed between a corresponding pair of second protrusions. A diffusion area is disposed between the at least one of the plurality of first protrusions and each of the corresponding pair of second protrusions. The at least one of the plurality of first protrusions has a cross-sectional area defined by the depth and the width of the first protrusion that is greater than the sum of the cross-sectional areas of the diffusion areas disposed between the at least one of the plurality of first protrusions and each of the corresponding pair of the second protrusions, the diffusion cross-sectional area being defined by the width and the height of the diffusion area. In some embodiments, the device further comprises an entry port disposed in communication with the first flow path. In some embodiments, the device further comprises an exit port disposed in communication with the second flow path. In some embodiments, each of the protrusions have a width of at least 1 μm and a depth of at least 20 μm. In some embodiments, each of the plurality of first protrusions have an aspect ratio that allows each of the plurality of first protrusions to completely fill with a fluid. In some embodiments, each of the plurality of second protrusions have an aspect ratio that allows each of the plurality of second protrusions to completely fill with a fluid. In some embodiments, the second substrate is glass and the first substrate is silicon. In some embodiments, the device further comprises a plurality of first protrusions disposed between a corresponding pair of second protrusions. In some embodiments, the device further comprises a diffusion area disposed between each of the plurality of first protrusions and each of the corresponding pair of second protrusions. In some embodiments, the second substrate further comprises at least one electrode. In some embodiments, the second substrate further comprises at least two electrodes. In some embodiments, one of the electrodes is disposed in communication with the first flow path and one of the electrodes is disposed in communication with the second flow path.

In still other embodiments, the present invention provides a device comprising a first substrate having a first and second face and having a plurality of first diffusion areas in the first substrate, a second substrate having a first and second face and having a plurality of second diffusion areas in the second substrate, a third substrate having a first and second face, a first flow path, and a second flow path. The second face of the first substrate is proximate to the second face of the second substrate, the first face of the second substrate is proximate to the first face of the third substrate, the first flow path is proximate to at least one of the plurality of first diffusion areas and at least one of the plurality of second diffusion areas, and the second flow path is proximate to at least one of the plurality of first diffusion areas and at least one of the plurality of second diffusion areas. In some embodiments, one electrode is disposed in communication with the first flow path and one electrode is disposed in communication with the second flow path.

In still other embodiments, the present invention provides a device comprising a first substrate having a first face and a second substrate having a first face, wherein the first face of the first substrate is proximate to the second face of the second substrate, the first substrate having a first protrusion on the first face of the first substrate, wherein the first protrusion has first side, a second side, a depth, and a width, a first diffusion area having a width and a height disposed proximate to the first side of the first protrusion, and a second diffusion area having a width and a height, disposed proximate to the second side of the first protrusion. The first protrusion has a cross-sectional area defined by the depth and the width of the first protrusion that is greater than the sum of a cross-sectional area of the first diffusion area defined by the width and the height of the first diffusion area and a cross-sectional area of the second diffusion area defined by the width and the height of the second diffusion area.

In still other embodiments, the present invention provides for a device comprising a first substrate structure directly bonded to a second substrate structure, wherein the first substrate structure comprises single crystal silicon, and wherein the second substrate structure comprises glass and at least one diffusion area disposed between the first and second substrate structures having a size less than about 500 nm and having a diffusion area uniformity of about ±1 nm to about 3 nm. In some embodiments, the diffusion area size comprises a height. In some embodiments, the diffusion area size is between about 3 nm to about 100 nm. In some embodiments, each of the first protrusions have a ratio of width to depth that allows each of the first protrusions to completely fill with a fluid.

In still other embodiments, the present invention provides for a device comprising a first substrate having a first face and a second substrate having a first face. The first face of the first substrate is proximate to the first face of the second substrate. The first substrate comprises a first flow path having a plurality of first protrusions on the first face of the first substrate, a second flow path having a plurality of second protrusions on the first face of the first substrate, and at least one diffusion area connecting at least one of the first protrusions to at least one of the second protrusions. At least one of the plurality of first protrusions is disposed between a corresponding pair of second protrusions. The second substrate comprises glass. In some embodiments, the device further comprises at least one anchor point and at least one spacer on the first face of the first substrate disposed such that the first face of the second substrate is bonded to the at least one anchor point and the at least one spacer. In some embodiments, the protrusions are rectangular. In some embodiments, the device comprises a plurality of anchor points and spacers. In some embodiments, the second substrate is selected to be one of translucent and transparent. In some embodiments, the glass is Pyrex 7740. In some embodiments, the first substrate is silicon. In some embodiments, the silicon is a double side polished single crystal silicon wafer. In some embodiments, the first substrate is bonded to the second substrate. In some embodiments, the first substrate is bonded to the second substrate by an anodic bond. In some embodiments, the device comprises a plurality of diffusion areas connecting at least one of the first protrusions to at least one of the second protrusions. In some embodiments, the device comprises a plurality of first protrusions disposed between a corresponding pair of second protrusions. In some embodiments, each of the first protrusions have a ratio of width to depth that allows each of the first protrusions to completely fill with a fluid. In some embodiments, the device comprises a capsule having a first and second capsule chambers wherein the device is disposed between the first and second chambers. In some embodiments, the device is disposed such that a substance in the first capsule path flows through the first and second paths to the second capsule path. The second capsule path has an opening disposed such that the substance can flow through the opening in the second capsule path. In some embodiments, the device further comprises an entry port disposed in communication with the first flow path. In some embodiments, the device further comprises an exit port disposed in communication with the second flow path.

In still other embodiments, the present invention provides a device comprising a first substrate having a first face and a second substrate having a first face. The first face of the first substrate is proximate to the first face of the second substrate. The first substrate comprises a first flow path having a plurality of first protrusions on the first face of the first substrate and a second flow path having a plurality of second protrusions on the first face of the first substrate. At least one of the plurality of first protrusions is disposed between a corresponding pair of second protrusions, the first flow path, and the second flow path are disposed such that a substance in the first flow path diffuses to the second flow path, and the first substrate comprises silicon and the second substrate comprises glass. In some embodiments, the second substrate comprises an entry port through the second substrate which aligns with first flow path of the first substrate. In some embodiments, each of the first protrusions have a ratio of width to depth that allows each of the first protrusions to completely fill with a fluid. In some embodiments, the diffusion is rate limiting.

In yet even further embodiments, the present invention provides a method for fabricating a device comprising etching at least one diffusion area, subsequently, etching a first flow path having a plurality of first protrusions and a second flow path having a plurality of second protrusions on a first face of a first silicon substrate, such that the at least one diffusion area is disposed between one of the first protrusions and one of the second protrusions, wherein the first and second protrusions have a depth and width and a cross-sectional area defined by the depth and width, wherein the at least one diffusion area has a length and a depth and cross-sectional area defined by the length and depth, and wherein the cross-sectional area of the first protrusion is greater than the cross-sectional area of the diffusion area. In some embodiments, the method further comprises the steps of masking the first and second flow paths prior to the step of etching the first and second flow paths and removing the mask subsequent to the step of etching the first and second flow paths. In some embodiments, the method further comprises the steps of masking the at least one diffusion area prior to the step of etching the diffusion area and removing the mask subsequent to the step of etching the at least one diffusion area. In some embodiments, the method further comprises anodically bonding a first face of a glass substrate to the first face of the first substrate. In some embodiments, the method further comprises providing an entry port in the glass substrate disposed to align with the first flow path. In some embodiments, the method further comprises etching an exit port aligned with the second flow path. In some embodiments, the step of etching at least one diffusion area comprises etching a plurality of diffusion areas. In some embodiments, the method further comprises growing an oxide in the etched at least one diffusion area to further define the at least one diffusion area.

In some embodiments, the present invention provides a device comprising a first substrate having a first face and a second substrate having a first face, wherein the first face of the first substrate is proximate to the first face of the second substrate. The first substrate comprises a first flow path having a plurality of first protrusions on the first face of the first substrate, a second flow path having a plurality of second protrusions on the first face of the first substrate, and a plurality of diffusion areas. At least one of the plurality of first protrusions is disposed between a corresponding pair of second protrusions. A diffusion area is disposed between at least one of the plurality of first protrusions and each of the corresponding pair of second protrusions. The second substrate comprises at least one electrode. In some embodiments, the second substrate further comprises at least two electrodes. In some embodiments, one of the electrodes is disposed in communication with the first flow path and one of the electrodes is disposed in communication with the second flow path.

In other embodiments, the present invention provides a device comprising a first substrate having a first face and a second substrate having a first face, wherein the first face of the first substrate is proximate to the first face of the second substrate. The first substrate comprises a first flow path having a plurality of first protrusions on the first face of the first substrate, wherein each of the plurality of first protrusions has a depth and a width, a second flow path having a plurality of second protrusions on the first face of the first substrate, wherein each of the plurality of second protrusions has a depth and a width, and a plurality of diffusion areas each of the plurality of diffusion areas having a length and a depth. At least one of the plurality of first protrusions is disposed between a corresponding pair of second protrusions. A diffusion area is disposed between the at least one of the plurality of first protrusions and each of the corresponding pair of second protrusions. The second substrate comprises at least one electrode. In some embodiments, the device further comprises an entry port disposed in communication with the first flow path. In some embodiments, the device further comprises an exit port disposed in communication with the second flow path. In some embodiments, each of the protrusions have a width of at least 1 μm and a depth of at least 20 μm. In some embodiments, the second substrate is glass and the first substrate is silicon. In some embodiments, the device further comprises a plurality of first protrusions disposed between a corresponding pair of second protrusions. In some embodiments, the device further comprises a diffusion area disposed between each of the plurality of first protrusions and each of the corresponding pair of second protrusions. In some embodiments, the second substrate further comprises at least two electrodes. In some embodiments, one of the electrodes is disposed in communication with the first flow path and one of the electrodes is disposed in communication with the second flow path. In some embodiments, the device further comprises an optical sensor. The optical sensor may be chosen from at least one of fluorescent oxygen sensor and flow sensor. In some embodiments, the device further comprises an electro-chemical sensor. The electro-chemical sensor may be chosen from at least one of glucose sensor, oxygen sensor, and carbon monoxide sensor. In some embodiments, the device further comprises a physics sensor. The physics sensor may be chosen from at least one of temperature sensor, pressure sensor, and flow sensor.

In still other embodiments, the present invention provides a device comprising a first substrate having a first and second face and having a plurality of first diffusion areas in the first substrate, a second substrate having a first and second face and having a plurality of second diffusion areas in the second substrate, a third substrate having a first and second face, a first flow path, and a second flow path. The second face of the first substrate is proximate to the second face of the second substrate, the first face of the second substrate is proximate to the first face of the third substrate, the first flow path is proximate to at least one of the plurality of first diffusion areas and at least one of the plurality of second diffusion areas, and the second flow path is proximate to at least one of the plurality of first diffusion areas and at least one of the plurality of second diffusion areas. At least one electrode is in the second substrate. In some embodiments, one electrode is disposed in communication with the first flow path and one electrode is disposed in communication with the second flow path.

In still other embodiments, the present invention provides a device comprising a first substrate having a first face and a second substrate having a first face, wherein the first face of the first substrate is proximate to the second face of the second substrate, the first substrate having a first protrusion on the first face of the first substrate, wherein the first protrusion has first side, a second side, a depth, and a width, a first diffusion area having a width and a height disposed proximate to the first side of the first protrusion, and a second diffusion area having a width and a height, disposed proximate to the second side of the first protrusion. At least one electrode is in the second substrate.

In still other embodiments, the present invention provides for a device comprising a first substrate structure directly bonded to a second substrate structure, wherein the first substrate structure comprises single crystal silicon, and wherein the second substrate structure comprises glass and at least one diffusion area disposed between the first and second substrate structures having a size less than about 500 nm and having a diffusion area uniformity of about ±1 nm to about 3 nm. At least one electrode is in the second substrate. In some embodiments, the diffusion area size comprises a height. In some embodiments, the diffusion area size is between about 3 nm to about 100 nm.

In still other embodiments, the present invention provides for a device comprising a first substrate having a first face and a second substrate having a first face. The first face of the first substrate is proximate to the first face of the second substrate. The first substrate comprises a first flow path having a plurality of first protrusions on the first face of the first substrate, a second flow path having a plurality of second protrusions on the first face of the first substrate, and at least one diffusion area connecting at least one of the first protrusions to at least one of the second protrusions. At least one of the plurality of first protrusions is disposed between a corresponding pair of second protrusions. The second substrate comprises glass. The glass substrate comprises at least one electrode. In some embodiments, the glass substrate comprises at least two electrodes. In some embodiments, the device further comprises at least one anchor point and at least one spacer on the first face of the first substrate disposed such that the first face of the second substrate is bonded to the at least one anchor point and the at least one spacer. In some embodiments, the protrusions are rectangular. In some embodiments, the device comprises a plurality of anchor points and spacers. In some embodiments, the second substrate is selected to be one of translucent and transparent. In some embodiments, the glass is Pyrex 7740. In some embodiments, the first substrate is silicon. In some embodiments, the silicon is a double side polished single crystal silicon wafer. In some embodiments, the first substrate is bonded to the second substrate. In some embodiments, the first substrate is bonded to the second substrate by an anodic bond. In some embodiments, the device comprises a plurality of diffusion areas connecting at least one of the first protrusions to at least one of the second protrusions. In some embodiments, the device comprises a plurality of first protrusions disposed between a corresponding pair of second protrusions. In some embodiments, the device comprises a capsule having a first and second capsule chambers wherein the device is disposed between the first and second chambers. In some embodiments, the device is disposed such that a substance in the first capsule path flows through the first and second paths to the second capsule path. The second capsule path has an opening disposed such that the substance can flow through the opening in the second capsule path. In some embodiments, the device further comprises an entry port disposed in communication with the first flow path. In some embodiments, the device further comprises an exit port disposed in communication with the second flow path.

In still other embodiments, the present invention provides a device comprising a first substrate having a first face and a second substrate having a first face. The first face of the first substrate is proximate to the first face of the second substrate. The first substrate comprises a first flow path having a plurality of first protrusions on the first face of the first substrate and a second flow path having a plurality of second protrusions on the first face of the first substrate. At least one of the plurality of first protrusions is disposed between a corresponding pair of second protrusions, the first flow path, and the second flow path are disposed such that a substance in the first flow path diffuses to the second flow path, and the first substrate comprises silicon and the second substrate comprises glass. At least one electrode is in the second substrate. In some embodiments, the device comprises at least two electrodes. In some embodiments, the second substrate comprises an entry port through the second substrate which aligns with first flow path of the first substrate. In some embodiments, the diffusion is rate limiting.

In yet even further embodiments, the present invention provides a method for fabricating a device comprising etching a first flow path having a plurality of first protrusions and a second flow path having a plurality of second protrusions on a first face of a first silicon substrate. Subsequently etching at least one diffusion area, such that said at least one diffusion area is disposed between one of said first protrusions and one of said second protrusions, etching at least one electrode area in a second substrate, forming an electrode in said electrode area, and depositing an oxide over said electrode. In some embodiments, the method further comprises the steps of masking said first and second flow paths prior to said step of etching said first and second flow paths and removing said mask subsequent to said step of etching said first and second flow paths. In some embodiments, the method further comprises the steps of masking said at least one diffusion area prior to said step of etching said diffusion area and removing said mask subsequent to said step of etching said at least one diffusion area. In some embodiments, the method further comprises anodically bonding a first face of a glass substrate to said first face of said first substrate. In some embodiments, the method further comprises providing an entry port in said glass substrate disposed to align with said first flow path. In some embodiments, the method further comprises etching an exit port aligned with said second flow path. In some embodiments, said step of etching at least one diffusion area comprises etching a plurality of diffusion areas. In some embodiments, the method further comprises growing an oxide in said etched at least one diffusion area to further define said at least one diffusion area.

Additional features and advantages of the invention will be set forth in part in the description that follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The following detailed description of embodiments of the present invention can be best understood when read in conjunction with the following drawings, where like structure is indicated with like reference numerals and in which:

FIG. 1 illustrates a cross-sectional view of a device.

FIG. 2 illustrates a top view of a device.

FIG. 3 illustrates a schematic three-dimensional view of a device with a glass top.

FIG. 4 illustrates a scanning electron microscope image of the first substrate.

FIGS. 5A-N illustrate a first substrate fabrication method in accordance with embodiments of the present invention.

FIG. 6 illustrates a schematic three-dimensional view of a device with a glass top and electrodes.

FIG. 7 illustrates a cross-sectional view of a device with electrodes.

FIG. 8A illustrates an implant assembly fitted with a device. The dashed arrow 414 represents a possible diffusion path of a molecule held within the device reservoir.

FIG. 8B illustrates an implant assembly having on board electronics and sensors.

FIG. 9A illustrates a cross-sectional view of a multilayer device.

FIG. 9B illustrates a cross-sectional view of a multilayer device taken 90° to FIG. 9A.

FIG. 10 illustrates a top view of a multilayer device.

FIGS. 11A-K illustrate a multilayer device fabrication method in accordance with embodiments of the present invention.

FIG. 12 illustrates glucose release curves for a passive device with 20 μm deep protrusions and nanochannels 50 nm in height.

FIG. 13 illustrates glucose release curves for a passive device with 30 μm deep protrusions and nanochannels 50 nm in height.

FIG. 14 illustrates lysozyme release curves for a non-passive device with 2 μm deep protrusions and nanochannels 50 nm in height.

FIG. 15 illustrates a portion of the device.

### DESCRIPTION OF THE EMBODIMENTS

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the embodiments herein is for describing particular embodiments only and is not intended to be limiting of the invention. As used in the description of the embodiments and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The invention generally relates to diffusion delivery systems and more particularly to high precision nanoengineered devices for therapeutic applications. The device contains diffusion areas that may be fabricated between bonded substrates, and the device can possess high mechanical strength. The invention further relates to capsules containing a diffusion delivery system. The present invention also relates to methods of fabricating the diffusion delivery systems.

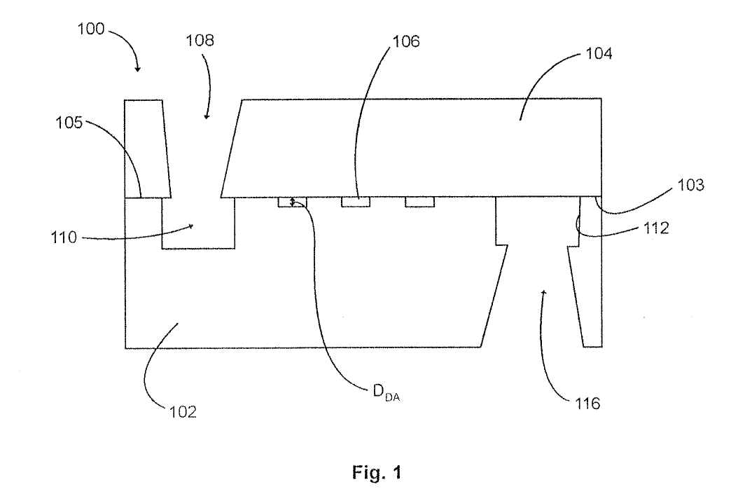

Referring to FIGS. 1-3, an embodiment of a device 100 is illustrated. The device 100 has a first substrate 102 having a first face 103 and a second substrate 104 having a second face 105. The first face 103 of the first substrate 102 is generally proximate to the first face 105 of the second substrates 104, and in some embodiments, the first substrate 102 can be bonded to the second substrate 104 in any suitable manner. For example, the first substrate 102 can be bonded to the second substrate 104 by anodic bonding.

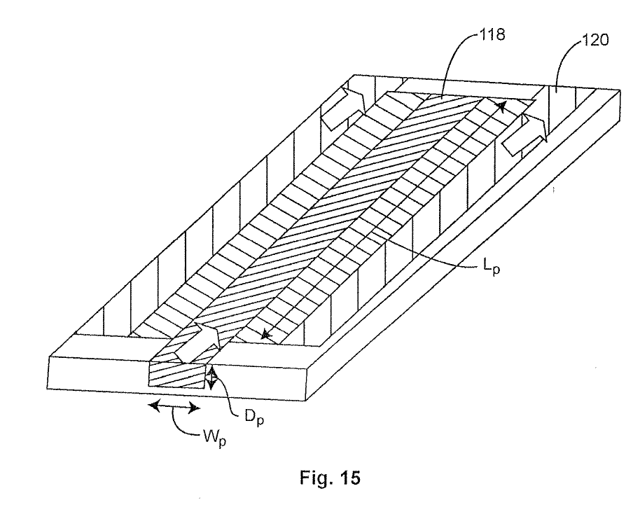

The first substrate 102 has a first flow path 110 having a plurality of first protrusions 118 and a second flow path 112 having a plurality of second protrusions 120 on the first face 103 of the first substrate 102. It will be understood that the term “on the first face” refers to a structure etched in the first face of a substrate or deposited on a first face of a first substrate. Each of the first protrusions 118 and the second protrusions 120 have a depth Dₚ, a width Wₚ, and length Lₚ and each of the first protrusions 118 and the second protrusions 120 have a cross-sectional area defined by the width Wₚ times the depth Dₚ of the protrusion 118 or 120 (FIG. 15). It will be understood that the protrusions 118 and 120 can be of any suitable dimensions. For example, the protrusions 118 and 120 can have a depth Dₚ of between about 1 μm to about 100 μm, or between about 5 μm to about 50 μm, or between about 10 μm to about 40 μm, or between about 20 μm to about 30 μm, or about 10 μm, or about 15 μm, or about 20 μm, or about 25 μm, or about 30 μm, or about 35 μm, or about 40 μm, a width Wₚ of between about 1 μm to about 500 μm, or between about 1 μm to about 250 μm, or between about 1 μm to about 100 μm, or between about 1 μm to about 50 μm, or between about 1 μm to about 25 μm, or between about 1 μm to about 10 μm, or between about 2.5 μm to about 10 μm, or between about 2.5 μm to about 7.5 μm, or about 1 μm, or about 2 μm, or about 3 μm, or about 4 μm, or about 5 μm, or about 6 μm, or about 7 μm, or about 8 μm, or about 9 μm, or about 10 μm, and a length Lₚ of between about 6 μm to about 5 mm, or between 10 μm to about 2.5 mm, or between about 25 μm to about 2 mm, or between about 100 μm to about 1.2 mm, or between about 0.5 mm to about 1.2 mm, or between about 1 mm to about 1.2 mm, or about 0.5 mm, or about 1 mm, or about 1.1 mm, or about 1.2 mm, or about 1.3 mm, or about 1.4 mm, or about 1.5 mm.

Referring now to FIG. 15, the aspect ratio of a protrusion 118, 120 is defined as the ratio of width Wₚ to the depth Dₚ of a protrusion 118, 120. It will be understood that the aspect ratio for a protrusion 118, 120 can be of any suitable ratio that allows the protrusion to fill with fluid. For example, the aspect ratio for a protrusion 118, 120 may be between about 1:1 to about 1:100, or between about 1:1 to about 1:50, or between about 1:1 to about 1:25, or between about 1:1 to about 1:20, or between about 1:1 to about 1:15, or between about 1:1 to about 1:10, or between about 1:1 to about 1:9, or between about 1:1 to about 1:8, or about 1:1 to about 1:7, or between about 1:1 to about 1:6, or between about 1:1 to about 1:5, or between about 1:1 to about 1:4, or between about 1:1 to about 1:3, or between about 1:1 to about 1:2, or between about 1:2 to about 1:20, or between about 1:2 to about 1:15, or between about 1:2 to about 1:10, or between about 1:2 to about 1:9, or between about 1:2 to about 1:8, or between about 1:2 to about 1:7, or between about 1:2 to about 1:6, or between about 1:2 to about 1:5, or between about 1:2 to about 1:4, or between about 1:2 to about 1:3, or between about 1:4 to about 1:6, or about 1:1, or about 1:2, or about 1:3, or about 1:4, or about 1:5, or about 1:6, or about 1:7, or about 1:8, or about 1:9, or about 1:10, or about 1:15, or about 1:20, or about 1:25, or about 1:50, or about 1:75, or about 1:100. It is also to be understood that the inverse of all the ratios recited may also allow the protrusion 118, 120 to completely fill with fluid, but would necessarily decrease the number of protrusions 118, 120 due to the increased width Wₚ of the protrusion as compared to the number of protrusions possible with the first recited set of aspect ratios. Any suitable number of first and second protrusions 118, 120 can be provided

Referring again to FIGS. 1-3, it will be understood that the first substrate 102 can comprise any suitable material. For example, the first substrate 102 can be silicon or a double side polished single crystal silicon wafer.

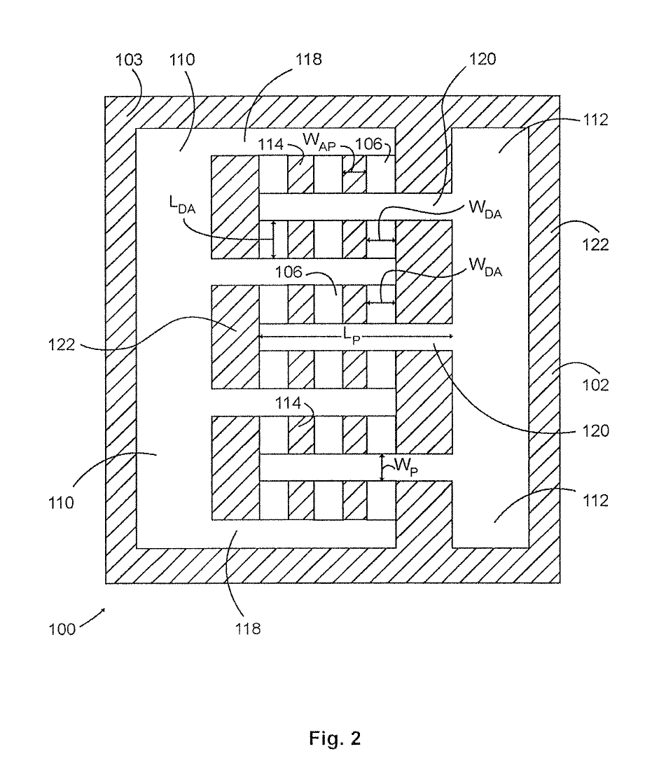

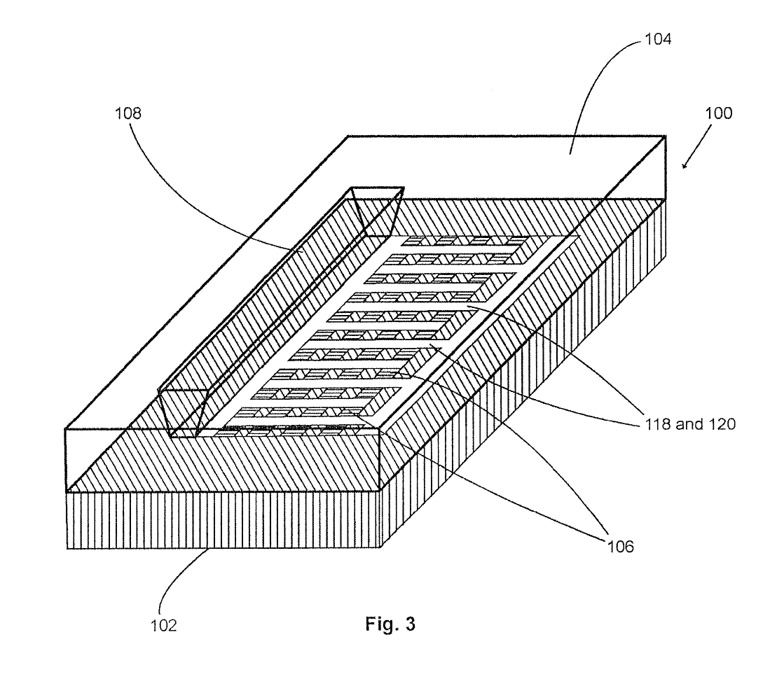

As illustrated in FIGS. 2-3, at least one of the first protrusions 118 may be disposed between a corresponding pair of second protrusions 120, and a plurality of first protrusions 118 can be disposed between a corresponding pair of second protrusions 120. However, it will be understood that such an arrangement is not necessary for the device to function. It will be understood that the first and second protrusions 118, 120 can be of any suitable shape. For example, the protrusions 118, 120 can be square, rectangular, circular, elliptical, tapered, triangular, or of any other suitable shape.

Referring now to FIGS. 1-3, the device 100 has diffusion areas 106 disposed on the first face 103 of the first substrate 102, and each of the diffusion areas 106 are disposed between a first protrusion 118 and a second protrusion 120. The diffusion areas 106 are further defined by the second substrate 104 as shown in FIG. 1. Each of the diffusion areas 106 have a length L_{DA}, a height H_{DA}, and width W_{DA} and each of the diffusion areas 106 has a cross-sectional area (not shown) defined by the height H_{DA} times the width W_{DA} of the diffusion area 106. When passive diffusion is used, the cross-sectional area of each of the first protrusions 118 is greater than the sum of the cross-sectional areas of the diffusion areas 106 disposed between the first protrusion 118 and the corresponding pair of second protrusions 120. In further examples, the cross-sectional area of each of the second protrusions 120 is greater than the sum of the cross-sectional areas of the diffusion areas 106 disposed between the second protrusion 120 and a corresponding pair of first protrusions 118. Without wishing to be bound, this area relationship is thought to allow the first flow path 110 to more easily fill with a substance, and maintain a constant diffusion rate, as described more fully herein.

The diffusion areas 106 may generally have any suitable dimensions. In one example, the diffusion areas 106 have dimensions on the nano-order. For example, the diffusion areas 106 can have a length L_{DA} of between about 1 μm to about 20 μm, or between about 1 μm to about 15 μm, or between about 1 μm to about 10 μm, or between about 2.5 μm to about 10 μm, or between about 5 μm to about 10 μm, or between about 2.5 μm to about 5 μm, or between about 5 μm to about 7.5 μm, or about 1 μm, or about 2 μm, or about 3 μm, or about 4 μm, or about 5 μm, or about 6 μm, or about 7 μm, or about 8 μm, or about 9 μm, or about 10 μm, a height H_{DA} of between about 1 nm to about 100 nm, or between about 1 nm to about 75 nm, or between about 1 nm to about 50 nm, or between about 1 nm to about 25 nm, or between about 1 nm to about 10 nm, or between about 10 nm to about 100 nm, or between about 10 nm to about 75 nm, or between about 10 nm to about 50 nm, or between about 10 nm to about 25 nm, or about 10 nm, or about 20 nm, or about 30 nm, or about 40 nm, or about 45 nm, or about 50 nm, or about 55 nm, or about 60 nm, or about 70 nm, or about 80 nm, or about 90 nm, or about 100 nm, and a width W_{DA} of between about 1 μm to about 5 mm, or between about 1 μm to about 4 mm, or between about 1 μm to about 3 mm, or between about 1 μm to about 2 mm, or between about 1 μm to about 1 mm, or between about 1 μm to about 0.5 mm, or between about 1 μm to about 0.25 mm, or between about 10 μm to about 100 μm, or between about 10 μm to about 75 μm, or between about 10 μm to about 50 μm, or between about 10 μm to about 25 μm, or between about 10 μm to about 15 μm, or about 5 μm, or about 10 μm, or about 11 μm, or about 12 μm, or about 13 μm, or about 14 μm, or about 15 μm, or about 16 μm, or about 17 μm, or about 18 μm, or about 19 μm, or about 20 μm, or about 25 μm, or about 50 μm, or about 75 μm, or about 100 μm, or about 0.25 mm, or about 0.5 mm, or about 1 mm, or about 2 mm, or about 3 mm, or about 4 mm, or about 5 mm. In some embodiments, the diffusion area width W_{DA} may be divided by an anchor point 114 resulting in multiple diffusion areas disposed in communication with a first protrusion and a second protrusion. The anchor point 114 may have a width W_{AP} of between about 1 μm to about 20 μm, or between about 1 μm to about 15 μm, or between about 1 μm and about 10 μm, or between about 1 μm to about 5 um, or between about 5 μm to about 10 μm, or between about 2.5 μm to about 7.5 μm, or between about 3 μm to about 7 μm, or between about 4 μm to about 6 μm, or about 1 μm, or about 2 μm, or about 3 μm, or about 4 μm, or about 5 μm, or about 6 μm, or about 7 μm, or about 8 μm, or about 9 μm, or about 10 μm. In another example, the diffusion area 106 can have a height H_{DA} of less than about 200 nm with a uniformity of about ±1 nm to about 3 nm. It will be understood that the diffusion areas 106 can comprise any suitable material. For example, the diffusion area 106 can be a nanochannel, multiple nanochannels, nano-porous materials, nanoporous forms, and/or any other option known to those skilled in the art.

Referring again to FIGS. 1-3, the second substrate 104 may have an entry port 108 that may be etched all the way through the second substrate 104 and may align with the first flow path 110 on the first substrate 102. It will be understood that the entry port 108 may have any suitable dimensions. For example, the entry port 108 can have dimensions of about 100 μm×3 mm, or about 200 μm×3 mm, or about 300 μm×3 mm, or about 350 μm×3 mm, or about 400 μm×3 mm, or about 500 μm×3 mm, or about 100 μm×2 mm, or about 200 μm×2 mm, or about 300 μm×2 mm, or about 350 μm×2 mm, or about 400 μm×2 mm, or about 500 μm×2 mm, or about 100 μm×1 mm, or about 200 μm×1 mm, or about 300 μm×1 mm, or about 350 μm×1 mm, or about 400 μm×1 mm, or about 500 μm×1 mm.

In addition, the device 100 may have an exit port 116 that aligns with the second flow path 112. It will be understood that the exit port 116 may have any suitable dimensions. For example, the exit port 116 can have dimensions of about 100 μm×3 mm, or about 200 μm×3 mm, or about 300 μm×3 mm, or about 350 μm×3 mm, or about 400 μm ×3 mm, or about 500 μm×3 mm, or about 100 μm×2 mm, or about 200 μm×2 mm, or about 300 μm×2 mm, or about 350 μm×2 mm, or about 400 μm×2 mm, or about 500 μm×2 mm, or about 100 μm×1 mm, or about 200 μm×1 mm, or about 300 μm×1 mm, or about 350 μm×1 mm, or about 400 μm×1 mm, or about 500 μm×1 mm.

The second substrate 104 can comprise any suitable substrate. For example, the second substrate 104 can comprise polysilicon. In some embodiments, the second substrate 104 may be a translucent or transparent glass. For example, the second substrate 104 can be Pyrex 7740 glass. Without intending to be bound, it is believed that the glass second substrate 104 increases the mechanical strength of the device 100, and it is believed that the bond between the first silicon substrate 102 and the second glass substrate 104 has increased bond strength. Additionally, a glass second substrate 104 allows the device 100 to be effectively visualized under a scanning electron microscope.

The device 100 may include spacer regions 122 along the edges and anchor points 114 at places between the diffusion areas 106, and the first substrate 102 may be bonded to the second substrate 104 at the spacer regions 122 and the anchor points 114 in any suitable manner. It will be understood that any suitable configuration of anchor points 114 and spacer regions 122. It will also be understood that anchor points 114 and/or spacer regions 122 may be any suitable shape or dimension. First 118 and second 120 protrusions may open into the edges of the first 110 and second 112 flow paths, respectively.

The substance being delivered through the device 100 may come to the first flow path 110 in the first substrate 102 through the entry port 108 in the second substrate 104, pass to the first protrusions 118 of the first flow path 110, diffuse through the diffusion areas 106 to the second protrusions 120 and then to the second flow path 112. The exit port 116 that may be aligned to the second flow path 112 in the first substrate 102 may provide a means for the substance to leave the device 100. It will be understood that any suitable substance can diffuse through the device in this manner. For example, water, glucose, lysozyme and FITC-BSA can diffuse through the device 100. Any other suitable drugs or substances can diffuse through this device. Spacer layers 124 can be provided at the ends of the protrusions 118 and 120 to close the protrusions 118 and 120 so that substances can diffuse through the diffusion areas 106.

In passive diffusion, the diffusion area 106 height H_{DA} may define the delivery rate limit and/or volume of the device 100. It will be understood that the effective porosity of the device may depend upon the number, height H_{DA}, and width W_{DA} of the diffusion areas 106, the width W_{AP} and periodicity of the anchor points 114, and/or the diffusion area 106 height H_{DA}. It will be understood that these geometries may be changed to design a device 100 having a desired flow rate and/or volume. It will be understood that flow rate and/or volume control may also be achieved by altering the aspect ratio of the first protrusion to the diffusion areas. It will be further understood that the diffusion area 106 height H_{DA} may result in diffusion having a linear rate. In one example, the overall device dimensions may be chosen to be about 4 mm×about 3 mm×about 1 mm.

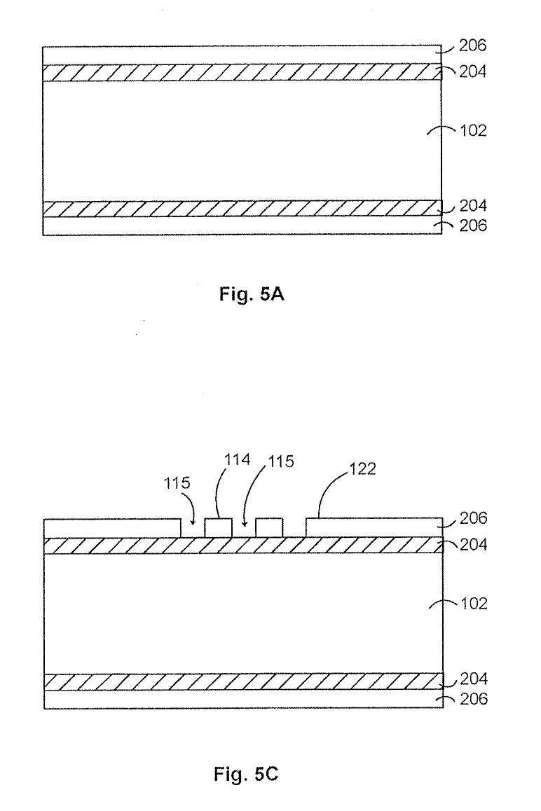

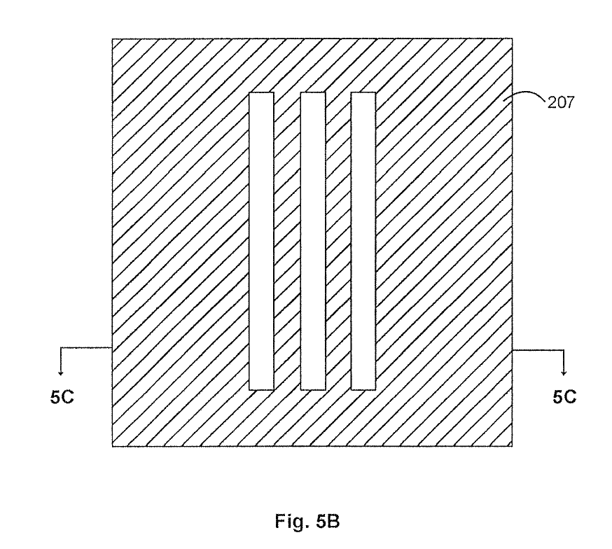

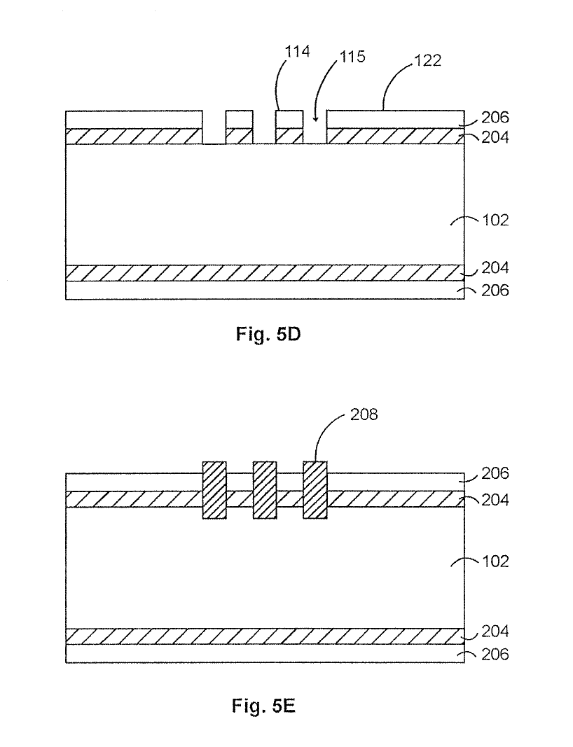

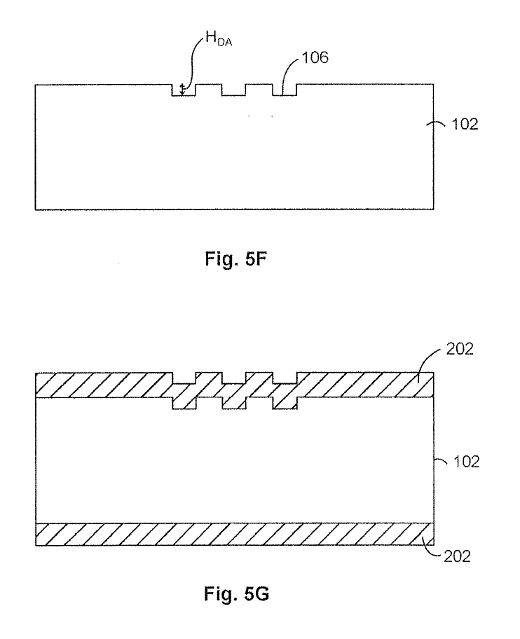

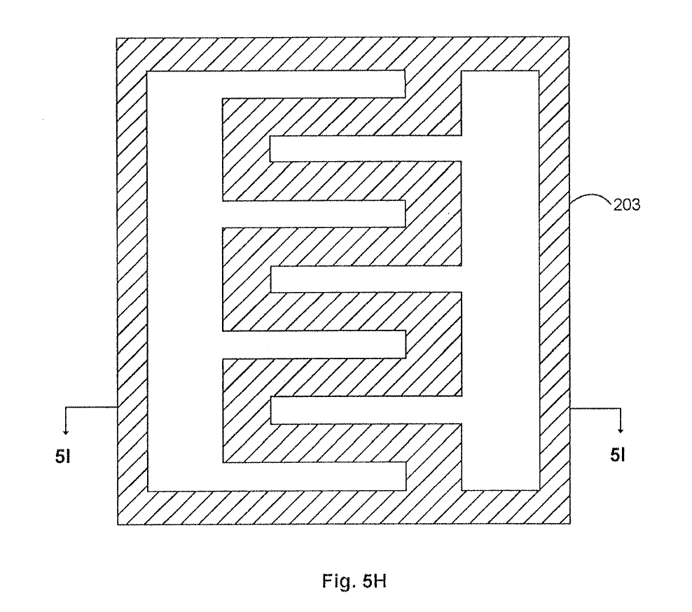

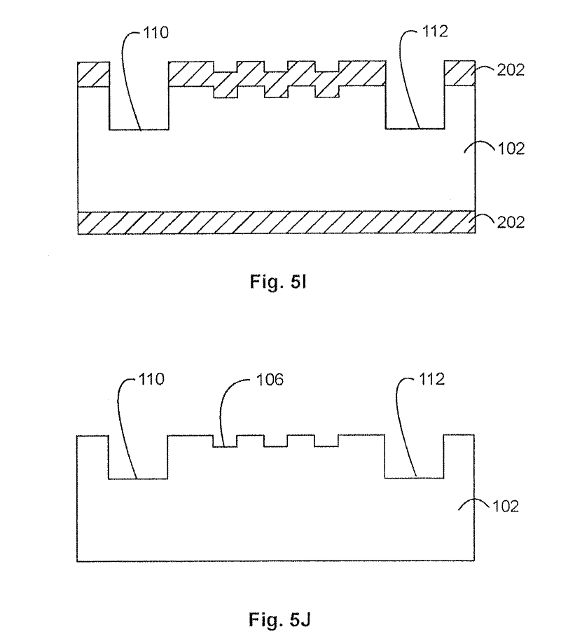

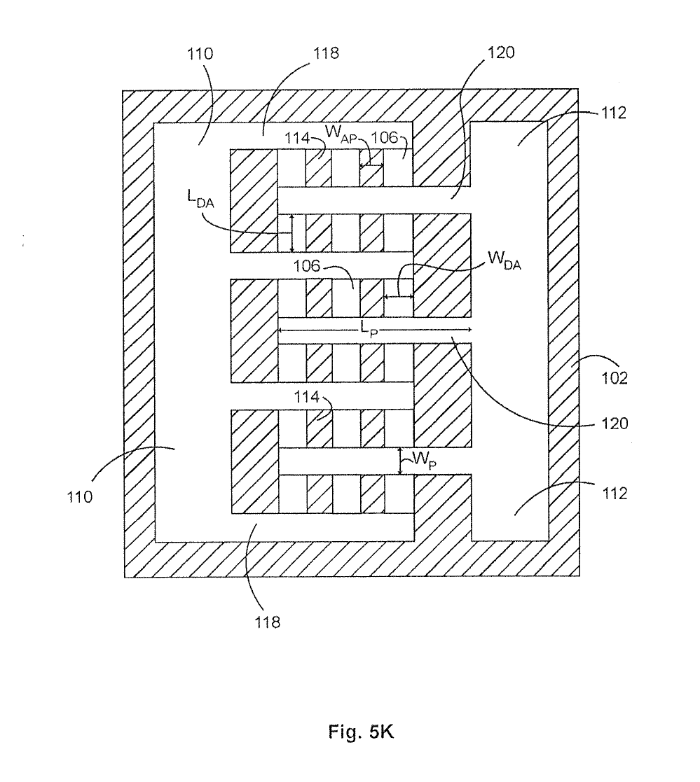

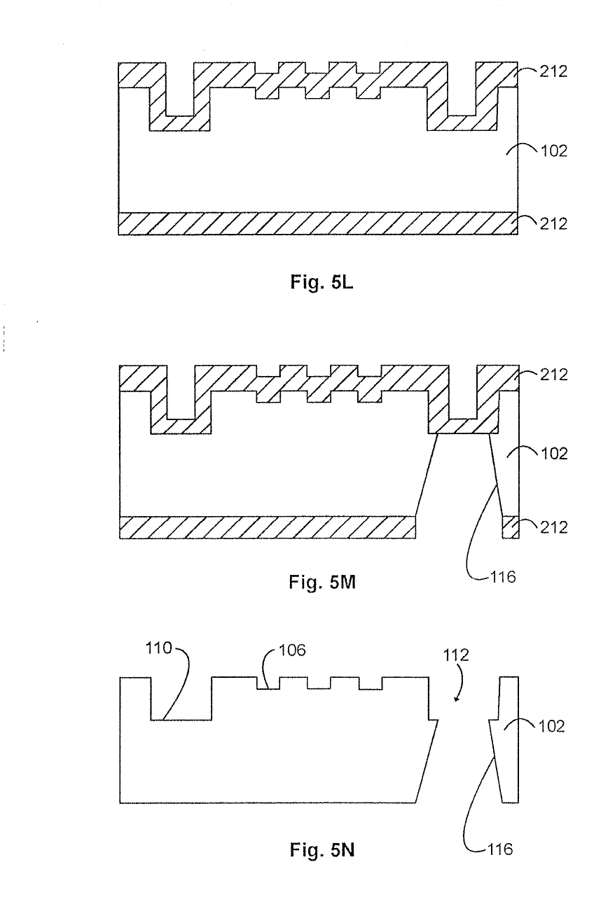

It will be understood that the device 100 can be formed in any suitable manner using any suitable methods. An exemplary fabrication process is described as following: a pad oxide layer 204 can be grown on the substrate 102 as shown in FIG. 5A. Additionally, a nitride layer 206 can be deposited on the pad oxide layer 204. The nitride layer 206 can be deposited by low stress low pressure chemical vapor deposition. A mask 207, as shown in FIG. 5B, can be provided to define the diffusion areas 106 and the spacer regions 122 and anchor points 114. The nitride layer 206 is etched in the areas defined by the mask 207 to the pad oxide layer 204, as shown in FIG. 5C.

Subsequently, the pad oxide layer 206 is selectively stripped versus silicon, as shown in FIG. 5D to form openings 115 and define anchor points 114 and spacer regions 122. Next a thermal oxide layer 208 is grown to the desired thickness as a sacrificial oxide layer 208, as shown in FIG. 5E. The height of the thermal oxide layer 208 controls the height of the diffusion area 106. The diffusion area 106 height may be defined as h=0.46 tₒₓ. Then the pad oxide layer 206, nitride layer 204, and sacrificial oxide layer 208 arc removed, as shown in FIG. 5F. Diffusion areas 106 are formed, and the diffusion areas 106 have a height H_{DA}. These layers may be removed in any suitable manner. For example, they can be removed using a low concentration HF solution.

As shown in FIG. 5G, an oxide mask layer 202 can be deposited on the first substrate 102 in any suitable manner. For example, the oxide mask layer 202 can be deposited by means of low pressure chemical vapor deposition (LPCVD). As shown in FIG. 5H, a mask 203 can be provided that defines the first and second flow paths 110, 112 and the first and second protrusions 118, 120 (not shown). The first and second flow paths 110, 112 and the first and second protrusions 118, 120 can be etched using any suitable etch, as shown in FIG. 51. For example, a KOH wet etching, a He+CHF₃+CF₄ plasma etch, an inductively coupled plasma etch, or a deep reactive ion etch can be used to reach a desired etch depth. The oxide mask 202 can be subsequently stripped in a HF solution, or Buffered Oxide Etcher, as shown in FIG. 5J. It will be understood that the mask material 202 could alternatively also be photoresist. It will be further understood that any suitable strip may be employed to remove the oxide mask 202.

A top view of FIG. 5J is shown in FIG. 5K. The first and second protrusions 118, 120 each have a width W_{P} and a length L_{P}. The diffusion areas 106 each have a width W_{DA} and a length L_{DA}. Anchor points have a width W_{AP}. Next, another nitride layer 212 is deposited over the top and bottom of the substrate 102, as shown in FIG. 5L. Subsequently, a mask is provided and an exit port 116 is etched on the bottom of the substrate 102, as shown in FIG. 5M. Finally, the nitride layer 212 is removed, as shown in FIG. 5N.

The second substrate 104 can have an entry port 108 provided in any suitable manner. For example, the second substrate 104 can have an entry port 108 drilled into the glass substrate 104. The first substrate 102 can be bonded in any suitable manner to the second substrate 104 after the first and second substrates 102, 104 are fabricated. For example, the first substrate 102 can be anodically bonded to the second substrate 104.

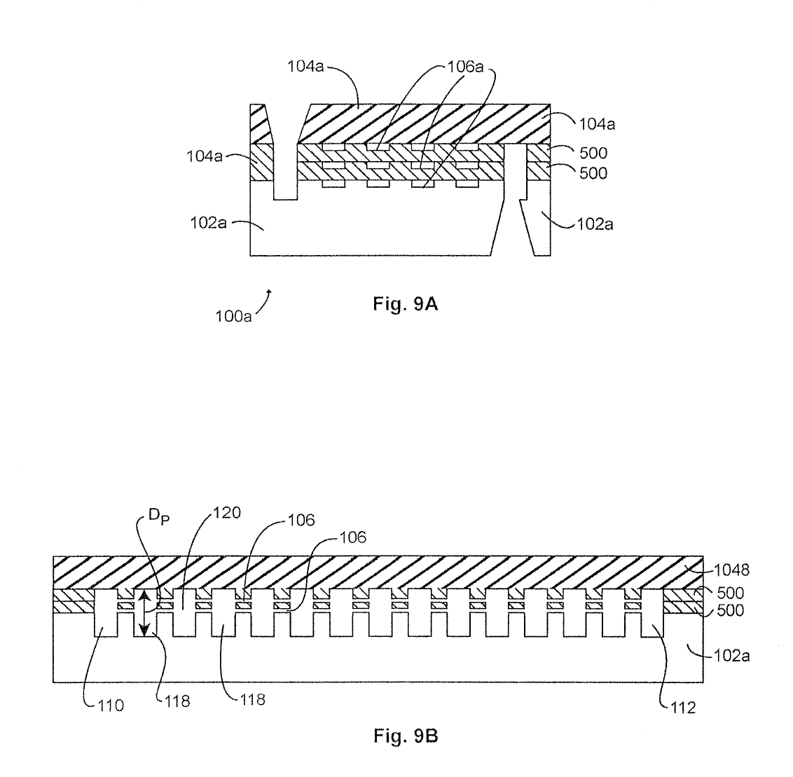

Referring now to FIGS. 9a, 9b, and 10 another embodiment of a device 100a is shown. The device 100a has one or more third substrates 500 disposed between the first and second substrates 102a, 104a. For example, device 100a may have one third substrate, or two third substrates, or three third substrates, or four third substrate, or five third substrates, or six third substrates, or seven third substrates, or eight third substrates, or nine third substrates, or ten third substrates, or more. Each of the first substrates have a first flow path 110a, a plurality of first protrusions 118 on the first face of the first substrate 102a and a second flow 112a path having a plurality of second protrusions 120 on the first face of the first substrate 102a. Each of the first and second protrusions 118, 120 have a depth Dₚ and a width Wₚ. The depth of the first and second protrusions 118, 120 is increased with each additional third substrate 500. Additionally, the first and third substrates 102a, 500 have a plurality of diffusion areas 106 and each of the plurality of diffusion areas 106 has a width, height, and length. At least one of the plurality of first protrusions 118 is disposed between a corresponding pair of second protrusions 120, and a diffusion area 106 is disposed between at least one of the plurality of first protrusions 118 and each of the corresponding pair of second protrusions 120. The first protrusions 118 have a cross-sectional area defined by the depth and width of the first protrusions 118 that is greater than the sum of the cross-sectional areas of the diffusion areas 106 disposed between the first protrusion 118 and the corresponding pair of second protrusions 120. The diffusion areas 106 have a cross-sectional area being defined by the width and height of the diffusion area, as discussed herein.

Multi-layer devices can increase the diffusion area by constructing a stack of many diffusion areas within a single protrusions. For example, a three-layer device has 3 times the total cross-sectional area of diffusion areas disposed between the first protrusion and the corresponding pair of second protrusions, compared to a single layer device. This multi-layer device allows a wide range of pre-defined porosity to achieve any arbitrary drug release rate using any preferred diffusion area size.

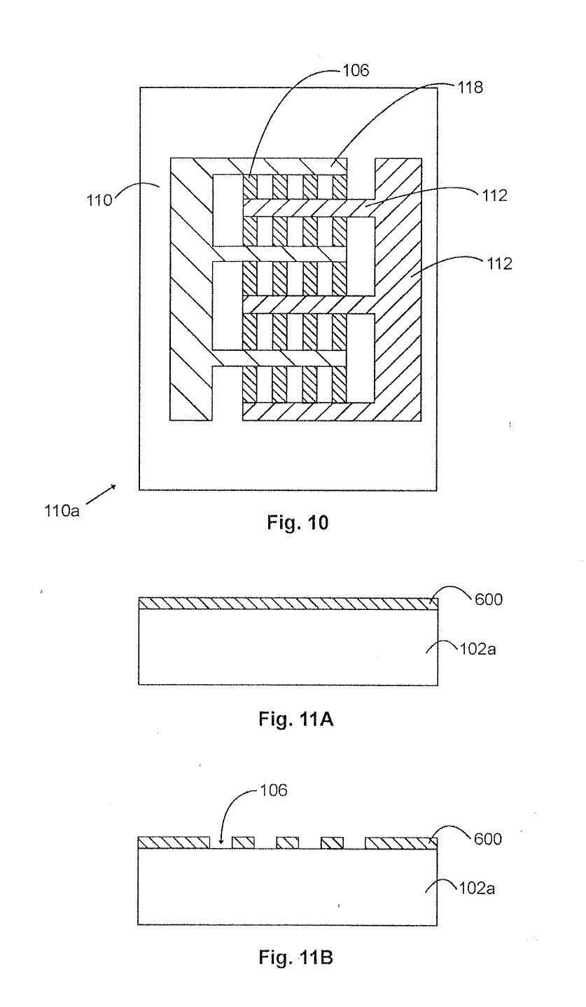

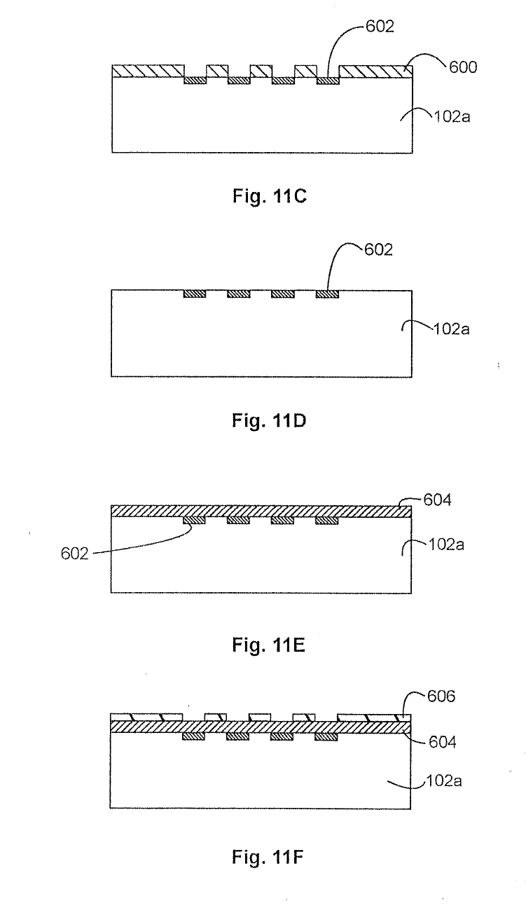

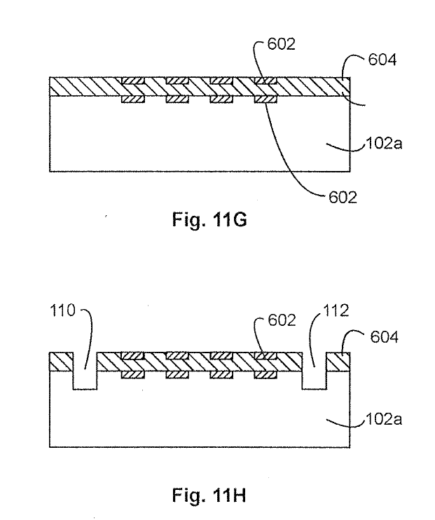

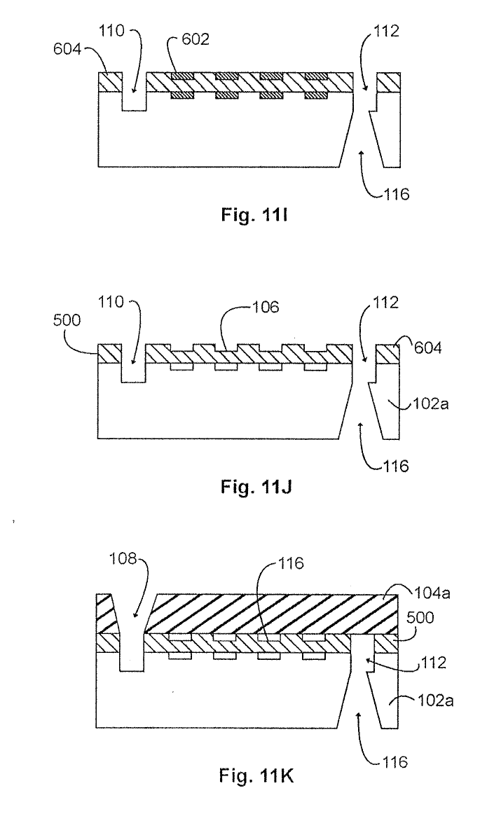

The microfabrication protocol consists of the following steps as seen in the FIGS. 11A-K. Starting with spare silicon wafer, 1) a thin hard mask layer 600, such as silicon nitride, is deposited on the first substrate 102a using LPCVD (FIG. 11A). 2) Then a standard photolithography process is used to define the diffusion area 106. 3) A dry etching process, such as RIE, is applied to remove nitride on the diffusion area. The photoresist is stripped, and the substrate 102a is cleaned (FIG. 11B). 4) Then a dry oxide sacrificial layer 602 is grown on the diffusion area 106. A two-step oxide growth may be applied to match the thickness of oxide layer 602 to the depth of diffusion area 106 (FIG. 11C). 5) Thereafter the nitride mask layer 600 is removed using phosphoric acid, which has high selectivity of nitride to silicon (FIG. 11D). Therefore, the oxide on diffusion area will not be removed. 6) A polycrystalline silicon film 604 with a thickness of several microns is then deposited by LPCVD. The silicon oxide 602 is buried under the polycrystalline silicon film 604. A chemical-mechanical polishing (CMP) process may be applied depending on the resulting surface flatness (FIG. 11E). 7) Then the dry oxide growth 606 on the diffusion area is repeated to get a second diffusion area layer (FIGS. 11F-11G). To get more layers of diffusion areas, the process is repeated from steps 1) to 6) until a desired number of layers are fabricated. Then a deep RIE is applied to define flow chambers 110, 112 and protrusions (not shown) (FIG. 11H). This RIE process also exposes buried oxide diffusion areas 602. Then a silicon nitride mask layer is deposited for deep KOH etching. The KOH wet etching produces exit ports from the backside of the substrate (FIG. 111). Then, the silicon nitride and silicon oxide is stripped, and diffusion areas are cleaned out (FIG. 11J). The second substrate 104a, which may be made of either silicon or glass with entry ports, is bonded on the third substrate 500 with multilayer diffusion areas (FIG. 11K). The bonded substrates are diced to get an individual multilayer device.

It will be understood that the internal dimensions of the devices 100, 100a may be optimized for high mechanical strength, so that the device is less likely to break in a subject if implanted. It is believed that these devices 100, 100a will possess high mechanical strength because the diffusion occurs at the interface of two bonded substrates. It will be further understood that bulk micro-fabrication technology may be used to fabricate these devices 100, 100a. With the use of a silicon dioxide sacrificial layer, diffusion areas 106 as small as 40 nm or less may be fabricated with size variations less than 4%.

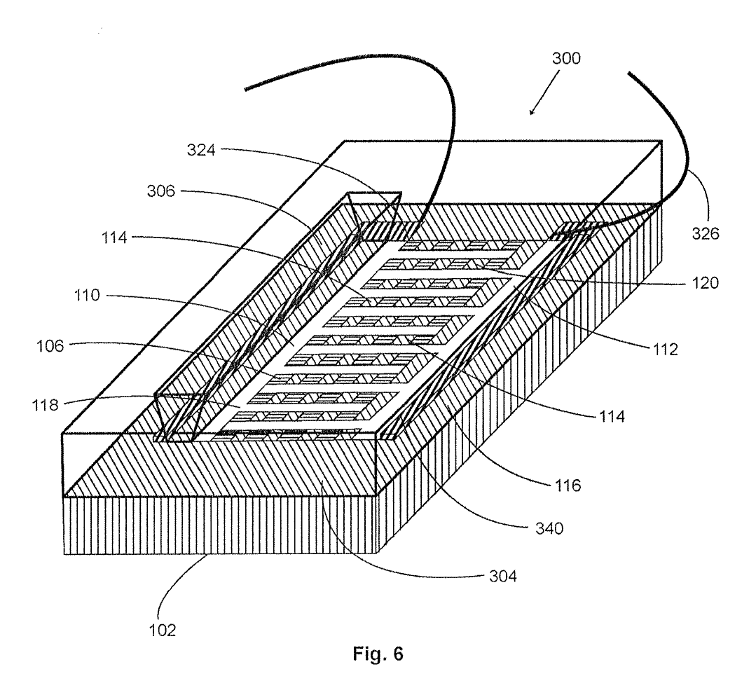

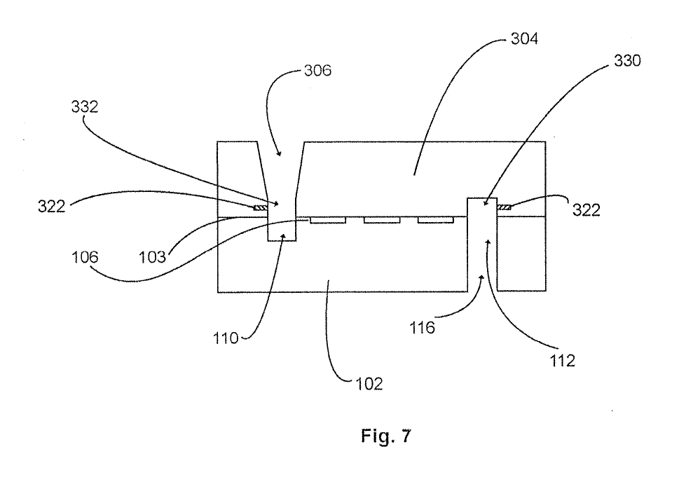

In accordance with other embodiments of the present invention, devices having electrodes that can be used to control diffusion rates of substances through the devices. Such a device 300 is illustrated in FIGS. 6 and 7. The device 300 has a first substrate 102 having features as already described herein. However, the second substrate 304 has at least one electrode 322 formed therein. For example, the second substrate 304 can have two electrodes formed therein. The electrodes 322 can be formed in any suitable manner from any suitable material. For example, the electrodes 322 can be formed from noble metals such as Pt, Ag, Au, Pd and Ir. In some instances, an intermediate layer such as Si₃N₄, SiO₂, Ti, or Ta layers (not shown) may be provided prior to the deposition of the electrodes 322 to promote adhesion of the electrodes 322 to the second substrate 304. Typical thicknesses of the adhesion layers and noble metal electrodes layers 322 can be on the order of about 0.05 μm and about 0.15 μm respectively. These electrode 322 metals may be deposited using evaporation or sputtering techniques. In another example, carbon may also been used as an electrode 322. Well adhering carbon thin film with good electrode properties may be obtained by either high temperature pyrolysis or by sputtering process in a DC or RF deposition mode.

Contact pads 324 can also be provided in the second substrate 304, and the contact pads 324 are areas that expose a portion of the electrode 322 so that a connection to the electrode 322 can be provided. In one example, the contact pads 324 are provided such that connecting wires 326 can be connected to the contact pads 324 at an edge of the second substrate 304. The electrodes 322 are disposed adjacent to first and second electrode contact chambers 332, 330, and the electrode contact chambers 332, 330 are disposed in communication with the first and second flow paths 110, 112. Therefore, the electrodes 322 can be in contact with a substance in the first and second flow paths 110, 112. The second substrate 304 also has an entry port 306 provided therein. The entry port 306 is disposed to align with the first flow path 110.

By applying voltage across these electrodes 322, the diffusion of a substance from the first flow path 110 through the diffusion areas 112 to the second flow path 112 may be controlled. For example, the electrodes 322 can be connected to an external pre-programmable circuit (not shown) that is programmed to apply voltages that allow manipulation of the diffusion rate. Therefore, the dosage rate of a substance can be controlled.

The electrodes 322 can be formed in any suitable manner. For example, openings in the second substrate 304 can be etched and the electrode 322 can be evaporated or sputtered onto the surface. The electrodes 322 can be patterned by photolithography accompanied by chemical etching (subtractive process) or lift-off (additive process). In chemical etching, a metal layer is first deposited. Electrode 322 areas may be photolithographically defined and then wet or dry etching may be performed to remove the metal from unwanted areas. Photoresist is usually spin-cast, but it can be sprayed-coated on the side-walls and at the bottom of etched electrode area grooves to obtain a uniform layer.

Lift-off has been used to pattern noble metals for which no etch process is compatible with photoresist masking. Examples of such metals are Pt, Ir or Pd. In the case of lift-off, electrode 322 areas may be photolithographically defined, followed by electrode metal deposition. By dissolving the underlying photoresist in an appropriate solvent, unwanted metallic parts may be lifted off, leaving the desired pattern on the surface. A high aspect ratio of photoresist and thin film electrode may be required for a successful pattern transfer. Vertical separation must be sufficient to prevent the metal deposition from becoming a continuous film. Pretreatment of photoresist has been suggested to form overhangs in order to achieve better lift-off. This process may involve soaking a prebaked photoresist in an aromatic solvent (e.g. chlorobenzene) before or after the exposure to UV-light. This overhang gives discontinuity between the metal layer deposited on the photoresist and that on the underlying layer or substrate, resulting in better defined electrode edge. An alternative to this is to use a two-layer resist structure. Different materials may be used for these two layers. A difference in development rate after exposure may cause an undercut in the bottom layer that ultimately forms an overhang in the top resist. This may be important for small inter-electrode spacing when a short circuit may result because of uncleaned electrode edges. Positive photoresists may be used more frequently since they dissolve in acetone easily. Even a carbon film may be patterned using plasma etching or lift-off.

In some cases a top passivation layer may be deposited on the top of these electrodes 322. It may consist of Si₃N₄ or SiO₂ deposited at low temperature. The deposition methods may be low pressure chemical vapor deposition (LPCVD) or plasma enhanced chemical vapor deposition (PECVD) processes. PECVD may be used when high temperature processes cannot be used as in the case of glass substrates 304 (the annealing point of Pyrex 7740 glass is 560° C. and the softening point is 821° C.). The top passivation layer may then be photolithographically patterned and etched to expose contact chambers 332 and 330 and the contact pads 324. The passivation layer can be subsequently polished until the surface of the substrate 304 is reached.

It will be understood that additional electronics or sensors can be provided in conjunction with the device 300. For example, sensors that sense the presence or absence of a certain molecule can be provided on the device 300, and the device 300 can be programmed to turn on the current to allow diffusion in response to such a sensor. Other sensors that can be incorporated include, but are not limited to, optical sensors such as fluorescent oxygen sensors and flow sensors, electro-chemical sensor such as glucose sensors, oxygen sensors, and carbon monoxide sensors, and physics sensors such as temperature sensors, pressure sensors, and flow sensors. The overall device 300 dimensions may be chosen to be any suitable dimensions. For example, the device 300 may be about 4 mm×3 mm×1 mm. The dimensions for the remaining features and components of device 300 are similar to those disclosed for device 100 herein. It will be understood that the aspect ratio of the first and second protrusions 118, 120 or the relationship of the cross-sectional area of the first protrusions 118 to the diffusion areas 106 are not necessarily important in providing a device 300 with desired diffusion rates because the electrodes 322 can have a voltage applied to provide a desired delivery rate.

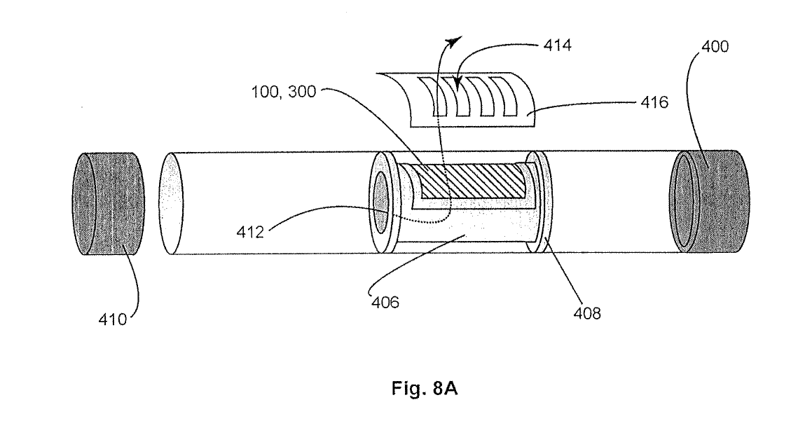

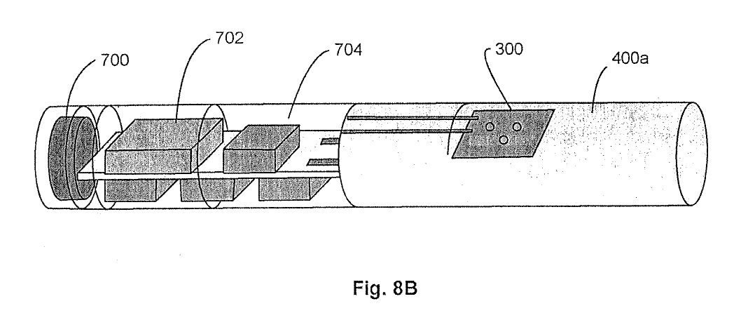

In accordance with further embodiments of the present invention, the devices 100, 100a, and 300 may be provided in a capsule for the purpose of implantation in the body. One such capsule 400 is illustrated in FIG. 8a. For example, the capsule 400 can be a cylindrical titanium capsule. One suitable implant assembly can be obtained from Manufacturing Technical Solutions (Carroll, Ohio). FIG. 8a shows a drawing of the implant 400 fitted with a device 100, 300. The device 100, 100a, and 300 may be affixed over a small-bore opening within a cylindrical methacrylate insert carrier 406 using general purpose silicone. This carrier 406 may be fitted with two rubber O-rings 408 at the ends. The completed carrier may be inserted into the titanium capsule until the device region is fully aligned under a grate 416 opening in the titanium capsule.

The devices 100, 100a, and 300 may divide the volume inside the capsule 400 into two chambers, with the only connection between the chambers being by flow through the device 100, 300. For example, the first chamber 412 can be a drug reservoir and the drug can diffuse through the grating 416 by diffusion through the device 100, 300. The substance may be contained in the chamber 412 below the carrier and device. The chamber above the device may be open to the exterior via the grate opening 416 of the capsule 400. Methacrylate end caps 410 containing re-sealable rubber septa may be used to seal the ends of the capsule 400 using silicone adhesive.

For filling a substance in the capsule, the capsule 400 may be oriented vertically and a 27 gauge luer-lock needle may be inserted into the upper septa for use as an air vent. A liquid suspension may be slowly injected into the implant via the lower septa until all the air within the implant is removed, as may be indicated by the presence of liquid exuding from the upper needle. The needles may be removed under gentle liquid injection pressure to avoid any concomitant influx of air upon withdrawal. The implants 400 may be rinsed by immersion in appropriate buffer prior to either placement into a testing vessel or surgical implantation. The small size of the capsule allows for relatively simple subcutaneous insertion in the arm or abdomen.

It will be understood that any suitable capsule can be used in conjunction with devices 100, 100a, and 300. For example, a capsule having first and second capsule paths can be provided, and the devices 100, 100a, or 300 can be disposed between the first and second capsule paths. The devices 100, 100a, or 300 can be disposed such that a substance in the first capsule path diffuses through the devices 100, 100a, or 300 into the second capsule path. Furthermore, a capsule 400a as shown in FIG. 8b can be used in conjunction with device 300. For example, the capsule in FIG. 8b can have sensors 702 that sense the presence or absence of a certain molecule, and the device 300 can be programmed to turn on the current to allow diffusion in response to such a sensor. With a control circuit 704 a battery 700 may also be included. Other sensors that can be incorporated include, but are not limited to, optical sensors such as fluorescent oxygen sensors and flow sensors, electro-chemical sensor such as glucose sensors, oxygen sensors, and carbon monoxide sensors, and physics sensors such as temperature sensors, pressure sensors, and flow sensors.

### EXAMPLES

### Example 1

### Passive Flow Device

### First Substrate Processing

Double side polished single crystal, 100 mm in diameter and 0.5 μm thick silicon wafer was used for first substrate fabrication. FIG. 5 shows the process flow for the first substrate fabrication. Nanochannels were defined and fabricated in the first step. The sacrificial oxide for the nanochannels can be grown thermally in a dry oxygen ambient with ±1% uniformity. The most common mask against such a local oxidation process is silicon nitride, which was used here. A pad oxide of 200Å thickness was first grown thermally by dry oxidation. The pad oxide reduces the stress between the silicon and silicon nitride layers and therefore enhances the adhesion of the two layers. A low stress LPCVD (low pressure chemical vapor deposition) nitride was then deposited using dichlorosilane (DCS) and NH₃ (100DCS/25NH3/140 mTorr/835° C.) on top of the pad oxide. The deposited nitride thickness was ˜2000Å. The nanochannel regions were defined photolithographically. The region between two diffusion areas is an anchor point where the second substrate bonds to the first substrate. The nitride layer was etched in the defined areas using He+SF₆ plasma. This etch was controlled so that the underlying pad oxide does not get etched so that the silicon surface is not etched. This is important in order to achieve good control of the nanochannel height. Then the pad oxide in the open areas was selectively (against silicon) etched in 1:10 HF:water solution. Once the silicon surface was exposed, a thermal oxide was grown to the desired thickness. This oxide growth defines the nanochannels size as mentioned earlier. Sacrificial oxide of thickness 109 nm was grown to give a 50 nm channel. Then the pad oxide, nitride, and sacrificial oxide layer were stripped in diluted HF solution.

The next step was the fabrication of the first flow path, second flow path, first protrusions and second protrusions. Low Temperature Oxide was used as a mask layer. This 0.5 μm thick oxide was deposited by LPCVD. The above-mentioned features were photolithographically defined using mask 1. The mask oxide was etched in the defined areas using a He+CHF₃+CF₄ plasma. The 30 um deep features were then etched into silicon using ICP. The mask layer nitride, underlying pad oxide, and the sacrificial oxide in nanochannel region were stripped afterwards in 1:10 diluted HF solution.

The final photolithography step for first substrate processing was for the exit port that was deep etched from the bottom side of this substrate. The exit port aligns to the second flow path. Another layer of LPCVD nitride was deposited (same deposition conditions). The deposited nitride thickness was ˜180 nm. This nitride protects the oxide in the nanochannel regions from being etched in the subsequent process. Backside photolithography was then performed to define the region of the exit port. The mask nitride was etched in the defined area using He+SF₆ plasma. This etch was performed until the silicon surface was exposed. A deep etch was then performed in 45 wt % KOH water solution heated at 80° C. The mask layer nitride was removed afterwards in diluted HF solution.

### Second Substrate Processing

Pyrex 7740 glass wafer, 100 mm in diameter and 0.5 μm thick wafer was used for the second substrate fabrication. The pattern of the entry port was ultrasonically drilled into this substrate.

### Substrate Bonding and Packaging

The glass second and silicon first substrate were bonded together using an anodic bonding technique. A mild bonding condition, such as 450 volts, 350° C., and 10 minute timing, was applied. The resulting bonding between silicon and glass was proven to have good bonding quality, and much stronger than the direct Si-Si bonding.

### Device Characterization

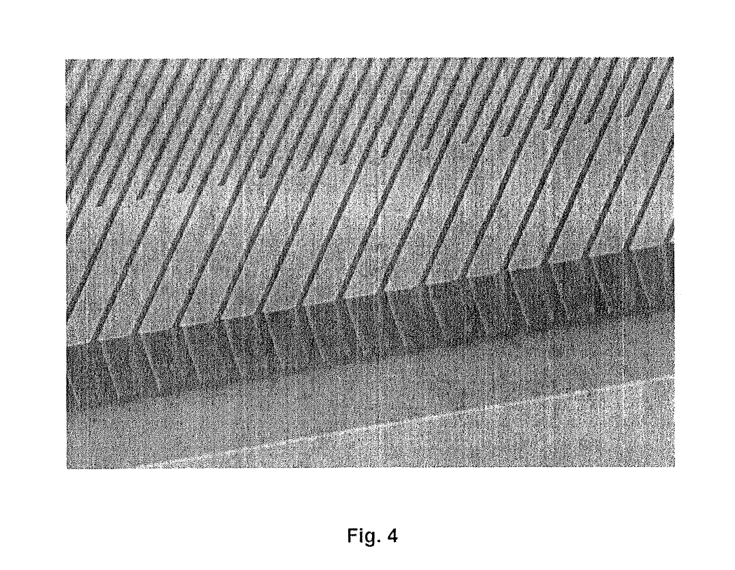

FIG. 4 shows an SEM (scanning electron microscopy) image of the first face of the first substrate, showing the protrusions and the spacer region. The nanochannels are between two protrusions (first and second protrusions), and each protrusion is blocked by a spacer region at the end. Once the second glass substrate is bonded to the silicon first substrate at the anchor points, the separation between the two substrates in between the two anchor points becomes a nanochannel.

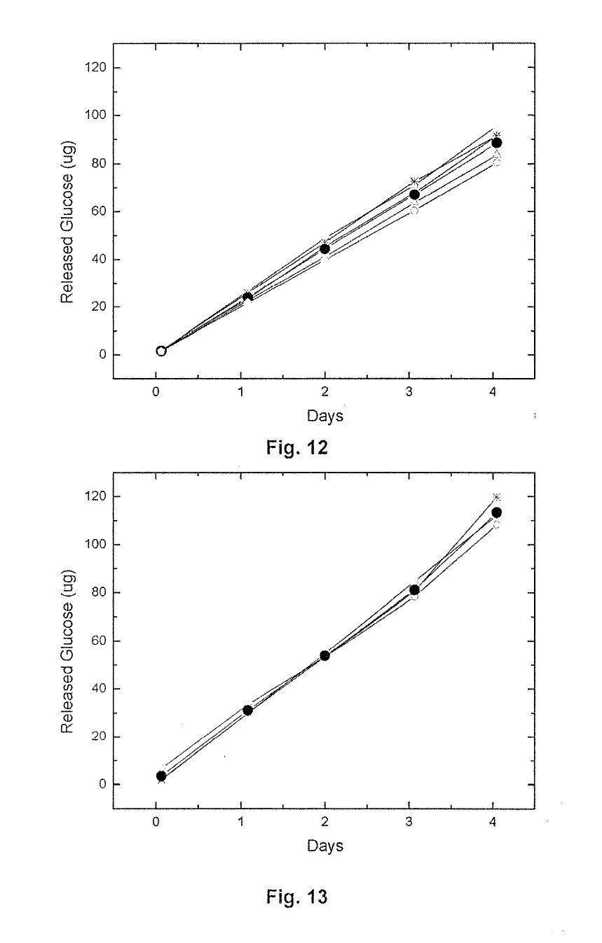

Diffusion characteristics of a passive flow device were investigated using glucose as the model molecule. The diffusion chambers were mounted on the tray of a plate shaker. The experiments were performed by applying 5 ml of a phosphate-buffered saline (PBS) solution, containing 0.2% of sodium azide, to the basolateral side of the diffusion chamber, and 0.20 ml of glucose solution (100 mg/ml) on top of it. An 8 mm diameter sphere was placed into the basolateral side of the well in order to make the solution homogeneous throughout the diffusion experiments. Plates were shaken at approximately 120 rpm. Samples were withdrawn at different time intervals and analyzed for the presence of glucose using The Amplexe® Red Glucose/Glucose Oxidase Assay Kit (Molecular Probes). Typical glucose release curves are shown in FIG. 12 for a passive device with 20 μm deep protrusions and nanochannels 50 nm in height, and in FIG. 13 a typical glucose release curve for a passive device with 30 μm deep protrusions and nanochannels 50 nm in height.

### Example 2

### Non-Passive Flow Device

### First Substrate Processing

The first substrate is cleaned in piranha by dipping the substrate for 10 minutes in a piranha bath. The first step is the fabrication of the first flow path, second flow path, first protrusions and second protrusions. 0.5 μm thick oxide is used as a mask layer. This oxide can be grown under the following conditions: H₂O ambient/1100° C./38 min.

Precise control of oxide thickness is not required here, since this oxide is used as a mask layer. The first flow path, second flow path, first protrusions and second protrusions are photolithographically defined using mask 1. The mask oxide is etched in the defined areas using a He+CHF₃+CF₄ plasma. Photoresist is later stripped off in piranha by dipping the substrates for 10 minutes. The above mentioned features are then etched into silicon using 45 wt % (by weight) KOH:H₂O solution heated at 70° C. The substrates are dipped in a KOH bath for 4 min 15 sec to achieve 2 μm deep features (first flow path, second flow path, first protrusions and second protrusions) in silicon. The mask oxide is then stripped in 49% HF solution by dipping the substrates in the bath for 10 minutes before proceeding to the next step. This 2 μm deep etch can also be achieved by a plasma etching method.

Nanochannels are defined and fabricated in the next step. The sacrificial oxide for the nanochannels is grown thermally in a dry oxygen ambient with ±1% uniformity. The most common mask against such a local oxidation process is silicon nitride, which is used here. A pad oxide of 600Å thickness is first grown thermally by dry oxidation. A thicker pad oxide is needed to achieve better control during subsequent nitride etching that uses timed etch. The following oxidation condition can be used for pad oxide growth: Dry oxidation/950° C./3 hr 20 min.

A stoichiometric nitride on top of the pad oxide is deposited as a mask.

The nanochannel regions are then defined photolithographically using mask 2. The nitride layer is etched in the defined areas using He+SF₆ plasma. This etch is controlled so that the underlying pad oxide does not get etched exposing the silicon. This is very important in order to achieve good control of the diffusion area height, since the end-point is based upon timed etch. Subsequently, the underlying pad oxide is selectively (against silicon) etched by dipping the wafers in 7:1 BHF. 7:1 BHF is chosen because of the process availability, while any BHF solution can be used for this purpose. Once the silicon surface is exposed in the diffusion area regions, a thermal oxide is grown to the desired thickness. This oxide growth defines the diffusion areas size. It is possible to achieve the oxide thickness within +/−1% thickness error by optimizing the time and temperature of the oxide growth.

The final photolithography step in the first substrate processing is for the formation of exit port and contact pad regions that are deep etched from the bottom side of this substrate. The exit port aligns to the second flow path. Backside photolithography is performed to define this region. The mask nitride and underlying pad oxide is etched in the defined area using He+SF₆ plasma. A controlled etch of nitride is not important here because the silicon underneath has to be etched all the way through in the subsequent step. So, this etch is performed until the silicon surface is exposed. A plasma etch is performed to achieve deep silicon etch. The mask layer nitride, underlying pad oxide, and the sacrificial oxide in diffusion area region are stripped afterwards in 49% HF solution.

### Second Substrate Processing

The second substrate is a glass substrate that contains electrodes, electrode contact chambers and entry port. The glass chosen here is Pyrex 7740 that has excellent bonding compatibility with silicon.

The first feature that is fabricated in glass is the electrodes. Lift-off is used for electrode formation. Grooves are etched into glass substrates deeper than the thickness of metal electrode and oxide is deposited after metal deposition to bury the metal electrode underneath the oxide. This is done in order to achieve good bonding between silicon and glass and to avoid metal electrodes/silicon contact that may cause another current path between the two electrodes. The deposited oxide also blocks any open path between the first flow path/second flow path and electrode contact chambers, and consequently prevents any fluid leakage. In order to achieve lift-off, two photolithography steps are used using two masks. Mask 2 has same features as on mask 1, but the features are 100 μm smaller (50 μm from each side) in each of x and y dimensions. This is done to avoid any metal deposition on the side walls of the etched regions and to avoid any metal deposition on the top surface of the glass substrate in case of any misalignment between mask 2 and mask 1.

To fabricate this structure, glass substrates are first cleaned in piranha solution. The lift-off regions are photolithographically defined using mask 1. These regions are 0.5 μm deep etched in a He+CHF₃+CF₄ plasma, and then the photoresist is stripped off in a piranha solution.

A second step photolithography is carried out to define the metal regions. Mask 2 is used for this purpose and is aligned with the alignment marks created during lift-off regions etch (mask 1). Please note that in this photolithography step, the substrates are not hard baked. Mask 2 opens up the regions where the electrode has to be deposited, while all other regions are still coated with the photoresist.

Titanium (Ti 0.05 μm)/Platinum (Pt 0.15 μm) is used as an electrode material. Electron-beam (e-beam) metal evaporator is used to deposit metal electrodes. Ti (0.05 μm)/Pt (0.15 μm) is deposited on the substrate surface. The metal gets deposited on the entire substrate surface. After metal deposition, the substrates are dipped in photoresist remover heated at 50° C. The photoresist remover is first heated in a beaker on a hot plate at 50° C. Substrates are transferred into the beaker, and then the beaker along with the substrate is transferred into the ultrasonic bath. Metal from the unwanted regions is lifted-off along with the photoresist.

One μm thick oxide is then deposited on this substrate. A PECVD process can be used to deposit the oxide. This allows oxide deposition at 200° C., as it is important to process the substrate below the glass transition temperature of Pyrex 7740. The 50 μm spacing between the deposited metal and the walls of etched ‘lift-off regions’ is wide enough for deposited oxide to fill conformally and to avoid any void formation. This is followed by a chemical mechanical polishing (CMP) step. CMP of deposited oxide is done until the glass surface is reached. A timed CMP is done to achieve this.

The next step is the fabrication of electrode contact chambers and contact pads. Photolithography is carried out using mask 3. The photolithographically defined regions are etched in a He+CHF₃+CF₄ plasma. The goal of this etch is to expose metal side walls in the electrode contact chambers. An overlap of 25 μm in electrode contact chamber over the metal electrode is in-built in mask 3 to assure the metal exposure in the electrode contact chambers. Further, the etch depth is kept more than the depth etched during mask 1 process. Metal exposure in this region is very important for establishing an electrokinetic flow in the device. 0.5 μm deep trenches are etched during mask 1 process; therefore 0.6 μm deep electrode contact chambers are etched here.

The last step is the fabrication of an exit port that is a deep etched all the way through the substrate from the back side of the glass substrate. This is done using an ultrasonic drilling technique.

### Substrate Bonding

The two substrates (silicon first substrate and glass second substrate) are bonded together so that the entry port in the second substrate is aligned with the first flow path in the first substrate. The bonding is achieved by anodic bonding method.

### Device Characterization

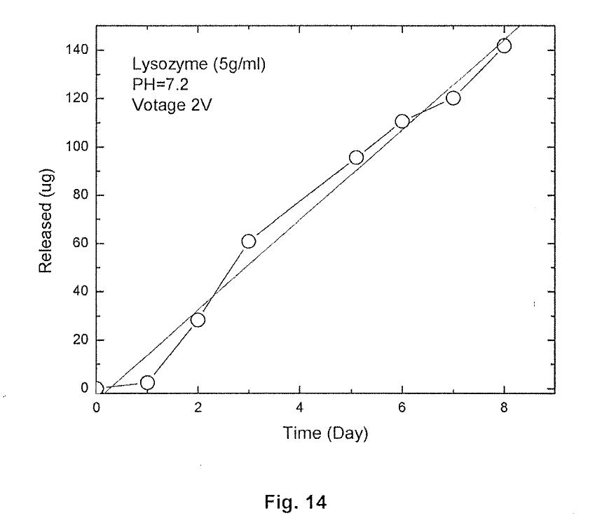

Diffusion characteristics of the non-passive flow device were investigated using lysozyme as the model molecule. The non-passive flow device was glued on a Costar Transwell diffusion chamber. The magnetic wires were bond to electrodes of the non-passive flow device, and the electrodes were then sealed with glue. The diffusion chambers were mounted on the tray of a plate shaker. The wires were connected to a DC power supply. The experiments were performed by applying 5 ml of a phosphate-buffered saline (PBS) solution, containing 0.2% of sodium azide, to the basolateral side of the diffusion chamber, and 0.20 ml of lysozyme solution (5 mg/ml) on top of it. An 8 mm diameter sphere was placed into the basolateral side of the well in order to make the solution homogeneous throughout the diffusion experiments. Plates were shaken at approximately 120 rpm. A 2 Volt voltage was applied constantly. Samples were withdrawn at different time intervals and analyzed for the presence of lysozyme using The EnzChek® Lysozyme Assay Kit (Molecular Probes). A typical lysozyme release curve is shown in FIG. 14 for a non-passive device with 2 μm deep protrusions and nanochannels 50 nm in height.

1-20. (canceled)

21. A device comprising:
a first substrate having a first flow path and a second flow path;
a second substrate proximate to the first substrate, wherein:
the first substrate and the second substrate define a diffusion area in communication with the first flow path and the second flow path; and
the diffusion area comprises multiple nanochannels;
a first electrode contact chamber in communication with the first flow path;
a second electrode contact chamber in communication with the second flow path;
a first electrode disposed adjacent the first electrode contact chamber, wherein the first electrode comprises a first contact pad;
a second electrode disposed adjacent the second electrode contact chamber, wherein the second electrode comprises a second contact pad;
a first electrical conductor coupled to the first contact pad; and
a second electrical conductor coupled to the second contact pad.

22. The device of claim 21 wherein the diffusion of a substance from the first flow path through the diffusion area to the second flow path may be increased by applying a current across the first and second electrodes.

23. The device of claim 21 wherein the first and second electrodes are connected to an external circuit programmed to apply currents to the first and second electrodes.

24. The device of claim 21 wherein the first flow path comprises a plurality of first protrusions and the second flow path comprises a plurality of second protrusions.

25. The device of claim 24 wherein the diffusion area is in communication with a first protrusion and a second protrusion.

26. The device of claim 21 wherein the first contact pad and the second contact pad are proximal to the device edge.
